(19) United States
(12) Patent Application Publication
(10) Pub. No.: US 2016/0129251 A1
(43) Pub. Date: May 12, 2016

(54) SYSTEMS AND METHODS FOR THE EXCHANGE OF DATA RELATED TO NONINVASIVE ELECTRICAL BRAIN STIMULATION

(71) Applicant: BRAINIQUE AG, Stans (CH)

(72) Inventors: Raymond Watt, San Diego, CA (US); Jaen Frederik Labuschagne, Cape Town (ZA); Michael J. Slater, Alpine, CA (US)

(73) Assignee: Brainique AG, Stans (CH)

(21) Appl. No.: 14/896,053

(22) PCT Filed: Jun. 9, 2014
(86) PCT No.: PCT/EP2014/061947

§ 371 Date: Dec. 4, 2015

Related U.S. Application Data
(60) Provisional application No. 61/832,737, filed on Jun. 7, 2013.

(51) Int. Cl. A61N 1/36; G06F 19/00; A61N 1/372

(52) U.S. Cl. CPC A61N 1/36025; A61N 1/37282; G06F 19/3425

(57) ABSTRACT

A computer-implemented method for sharing information related to a noninvasive electrical brain stimulation “NIEBS” treatment, comprising: providing a NIEBS treatment to a user via a NIEBS generator, said NIEBS treatment having variables; receiving adjustments to the variables of said NIEBS treatment via user input; and creating a NIEBS Control Profile based on said received adjustments.

### TECHNICAL FIELD

This application relates to systems and methods for the exchange of data related to noninvasive electrical brain stimulation.

### BACKGROUND

Noninvasive Electrical Brain Stimulation (herein referred to as NIEBS) applies gentle micro-current pulses to the brain using electrodes. It is widely accepted that NIEBS stimulates the brain to manufacture neurotransmitters. Noninvasive electrical brain stimulation has also been proposed for treatment of various medical conditions.

The signals operate to normalize the electrical output of the brain. NIEBS has thus been used/tested to treat substance dependence, depression and anxiety. It has been noted in at least some instances that NIEBS has equal or greater efficacy for the treatment of depression when compared to antidepressant medications, with fewer side effects.

The mechanism by which NIEBS produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

A user or patient of a NIEBS treatment may have a measure of control over the treatment.

### SUMMARY

According to a first aspect, there is provided a computer-implemented method for sharing information related to a noninvasive electrical brain stimulation “NIEBS” treatment, comprising: providing a NIEBS treatment to a user via a NIEBS generator, said NIEBS treatment having variables; receiving adjustments to the variables of said NIEBS treatment via user input; and creating a NIEBS Control Profile based on said received adjustments.

By creating a NIEBS Control Profile it is possible to relate the personal preferences of a user received a NIEBS treatment to enable attributes relating to the treatment to be stored in a simple manner. By relating the personal preferences to a NIEBS Control Profile, it is possible to re-use and share a user's personal adjustments to the NIEBS treatment in a simple and efficient manner.

According to a second aspect, there is provided a computer-implemented method for sharing information related to a noninvasive electrical brain stimulation “NIEBS” treatment, comprising: receiving a NIEBS Control Profile at a server; making said NIEBS Control Profile available to users for download; receiving information from a user relating to said NIEBS Control Profile; and making said information from said user available with said NIEBS Control Profile.

By making the NIEBS Control Profile available to users for download, it is possible to share a particular adjustment to a NIEBS treatment made by a user with other users that may use the same adjustment to adjust their own NIEBS treatment in a manner that is similar to that of the original user. By receiving information from a user relating to the NIEBS Control Profile and making that information available, it is possible for a user to provide information relating to a particular profile, for example in the form of feedback or a review, such that any further users may access the information relating to the NIEBS Control Profile and gain an understanding of the effect or benefit of a particular treatment adjustment before applying that adjustment.

According to a third aspect, there is provided a computer-implemented method for sharing information related to a noninvasive electrical brain stimulation “NIEBS” treatment, comprising: providing a NIEBS treatment to a user via a NIEBS generator; receiving adjustments to variables of said NIEBS via user controls; and creating a NIEBS Control Profile based on said adjustments.

By creating a NIEBS Control Profile it is possible to relate the personal preferences of a user received a NIEBS treatment to enable attributes relating to the treatment to be stored in a simple manner. By relating the personal preferences to a NIEBS Control Profile, it is possible to re-use and share a user's personal adjustments to the NIEBS treatment in a simple and efficient manner.

According to a fourth aspect, there is provided a computer-implemented method for sharing information related to a noninvasive electrical brain stimulation “NIEBS” treatment, comprising: receiving a NIEBS Control Profile at a server; making said NIEBS Control Profile available to users for download; receiving feedback from said users regarding said NIEBS Control Profile; and making said feedback from said user available with said NIEBS Control Profile.

By making the NIEBS Control Profile available to users for download, it is possible to share a particular adjustment to a NIEBS treatment made by a user with other users that may use the same adjustment to adjust their own NIEBS treatment in a manner that is similar to that of the original user. By receiving feedback from a user relating to the NIEBS Control Profile and making that feedback available, it is possible for a user to provide information relating to a particular profile, such that any further users may access the feedback for the NIEBS Control Profile and gain an understanding of the effect or benefit of a particular treatment adjustment before using that adjustment.

### BRIEF DESCRIPTION OF THE DRAWINGS

FIG. 1A illustrates a block diagram of an example environment for a noninvasive electrical brain stimulation system in accordance with embodiments of the present technology.

FIG. 1B illustrates a graphical interface showing adjustments made to a NIEBS treatment in accordance with embodiments of the present technology.

FIG. 1C illustrates a block diagram of an example environment for managing data for a noninvasive electrical brain stimulation system in accordance with embodiments of the present technology.

FIG. 2 illustrates block diagrams of example waveforms for use in a noninvasive electrical brain stimulation system in accordance with embodiments of the present technology.

The drawings referred to in this description of embodiments should be understood as not being drawn to scale except if specifically noted.

### DETAILED DESCRIPTION

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

### Overview of Systems and Methods for the Exchange of Data Related to Noninvasive Electrical Brain Stimulation

Embodiments of the present technology are for systems and methods for the exchange of data related to electro medicine. The description and claims herein specifically describe noninvasive electrical brain stimulation (NIEBS). However, the present technology applies generally to electrotherapy and electro medicine in its many forms. Therefore, the descriptions and claims related to NIEBS may be extended to include electrotherapy in general. Types of electrotherapy may be for, but are not limited to, electro neurostimulation, electro neuromodulation, neuromdulation, brain stimulation, electro medicine, bone growth, muscle stimulation, pain management, etc.

For example, neurostimulation involves modulation of the nervous system and electrically activate neurons in the body. The activation of neural elements in a part of the nervous system can be effectively facilitated by stimulation. Micro-electrodes are utilized to interface with excitable tissue in order to either restore recording experiences to the implant recipient or control an effector organ. Additionally, neuromodulation is the physiological process by which a given neuron uses several different neurotransmitters to regulate diverse populations of central nervous system neurons. This is in contrast to classical synaptic transmission, in which one presynaptic neuron directly influences a single postsynaptic partner. Neuromodulators secreted by a small group of neurons diffuse through large areas of the nervous system, affecting multiple neurons. Examples of neuromodulators include dopamine, serotonin, acetylcholine, histamine and others.

NIEBS is a treatment that applies pulses to the brain across the head of the patient using electrodes. There are many types of NIEBS such as transcranial direct current stimulation (tDCS) which is a form of neuro-stimulation which uses constant, low current delivered directly to the brain area of interest via small electrodes. There are different types of tDCS: anodal, and cathodal. The anodal stimulation is positive (V+) stimulation that increases the neuronal excitability of the area being stimulated. Cathodal (V−) stimulation decreases the neuronal excitability of the area being stimulated. Cathodal stimulation can treat psychological disorders that are caused by the hyper-activity of an area of the brain.

Another form of NIEBS is transcranial alternating current stimulation (tACS) which is a noninvasive means by which alternating currents applied through the skull over the occipital cortex of the brain entrains in a frequency-specific fashion the neural oscillations of the underlying brain. Another class of NIEBS is transcranial pulsed current stimulation (tPCS).

tPCS is a noninvasive method that employs a waveform for use in NIEBS. A tPCS generator is a self-powered device that implements either a fixed tPCS therapy program with preset parameters, or a programmable device that can receive a tPCS therapy program based on treatment options determined by a healthcare professional to be of use to a person with a specific condition. tPCS may also employ a chaotic system that varies many of the pulse characteristics in a random, non-repetitive process.

The present technology is not limited to one form of NIEBS. Therefore, as used herein, NIEBS may refer to many varieties of NIEBS including, but not limited to, transcranial direct current stimulation (tDCS), transcranial alternating current stimulation (tACS), tPCS, and any other neuro-stimulation type protocols such as random noise stimulation and chaotic noise stimulation.

NIEBS involves brain stimulation by low current low voltage that may use alternating square waves or other waves. The effect is to improve the brain's “plasticity,” making it easier to learn. The effect may also be described as an increase in focus, getting into the flow, or being in the zone.

The present technology employs hardware for NIEBS that attaches electrodes to the head of the patient. The hardware may also include speakers such as headphones. The present technology may apply NIEBS to a user and may or may not simultaneously play audio for the user via speakers such as headphones. The pulse for the NIEBS may or may not be based on the rhythm or beat of the audio signal. The speakers may be separate from the electrodes or may be combined into one frame or housing with the electrodes.

The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

The NIEBS treatment, or other electro medicine, may be adjusted, modified or controlled by a user or patient. For example, a user may control the intensity of amplitude of the treatment. The user may develop levels of control that are preferred by the user. Such levels of control may be described as a NIEBS Control Profile. The user may wish to share her NIEBS Control Profile with other users or share other information regarding a NIEBS Control Profile. Other information may be reviews, feedback, or blogs regarding NIEBS Control Profiles. For example, a user may post a NIEBS Control Profile with a review of the profile. A second user may then download the NIEBS Control Profile and offer feedback or comments. Such forums may be public or private.

Embodiments for Systems and Methods for the Exchange of Data Related to Noninvasive Electrical Brain Stimulation

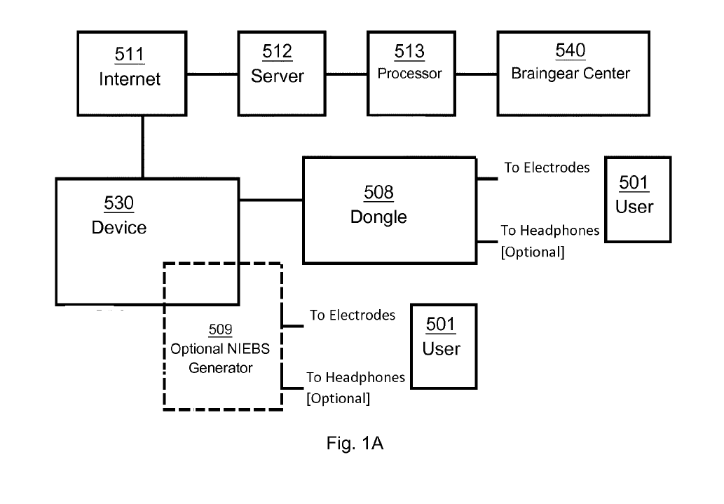

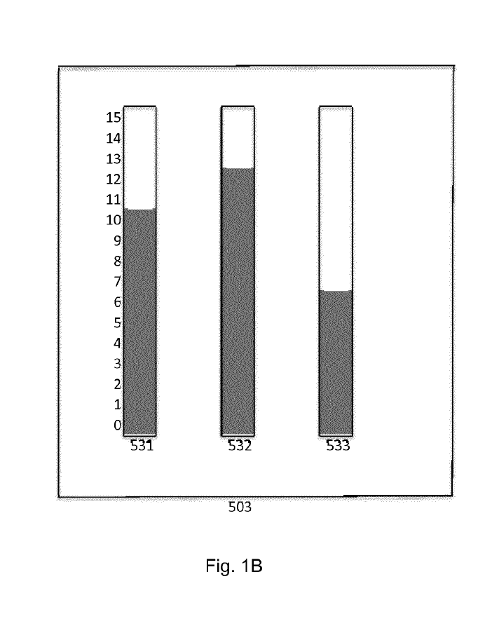

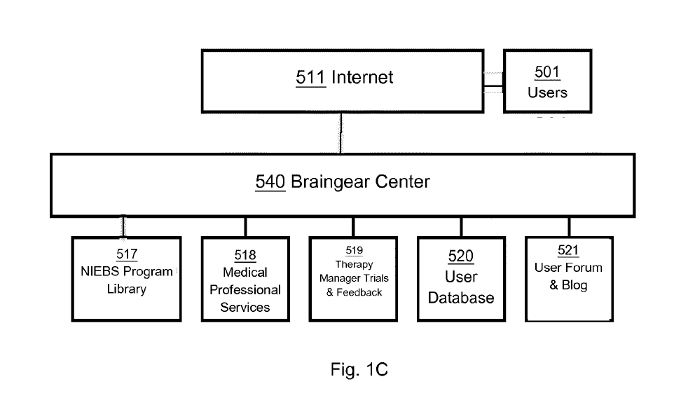

With reference to FIG. 1A which is a depiction of an ecosystem for managing NIEBS or other electro medicine. FIG. 1A relates to user connection to the Braingear Ecosystem. Device 530 may be a personal digital assistant, digital media player, smartphone, handheld electronic device, or may also be a computer system such as a desktop, laptop, netbook, tablet, etc. The device 530 may access Internet Internet 511 via a cell phone or Wi-Fi connection. Through the Internet or other network connection, the device 530 may download or upload data to and from server 512 and processor 513. Braingear center 540 is a database or a repository for data associated with NIEBS. In one embodiment, Braingear center 540 comprises server 512 and processor 513. For example, Braingear center 540 may comprise NIEBS Control Profiles, power tunes, the ability to combine audio tracks with power tunes, prescriptions, user ratings and reviews of power tunes or other items.

NIEBS treatment may be provided to user 501 via a built in NIEBS generator such as 509 or via a separate hardware device coupled with device 530 such as dongle 508. Dongle 508 and NIEBS generator 509 are able to generate NIEBS treatment signals that are used to provide NIEBS treatment to a user via electrodes. Device 530 may also be able to play music for user 501 via headphones or other speaker simultaneous to the NIEBS treatment. The music may be relayed to the user via dongle 508.

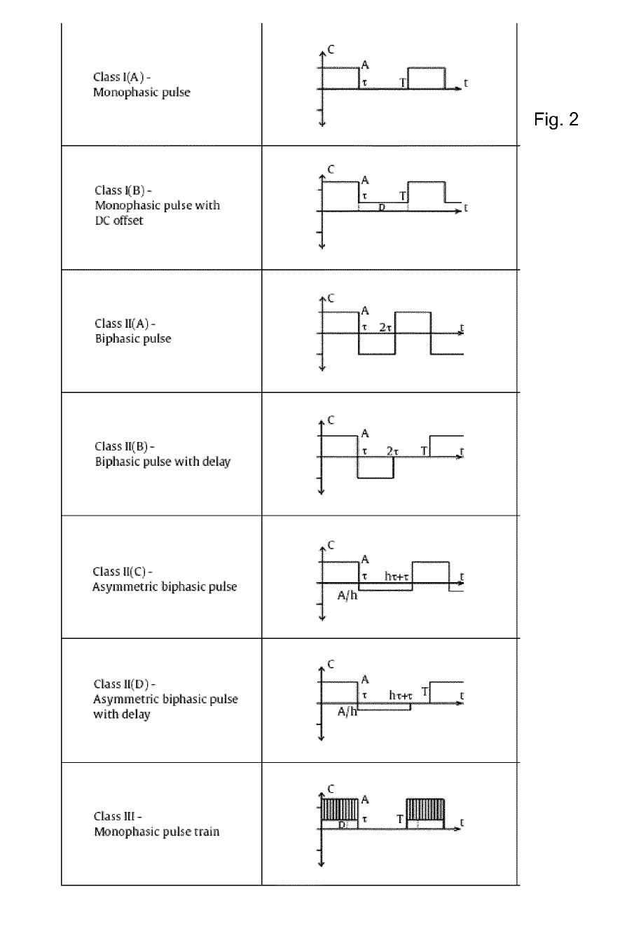

In one embodiment, the device 530 is an off the shelf product with no NIEBS generation abilities. The device 530 is associated with user 501 and may be personalized or customized for user 501. The NIEBS signal for NIEBS treatment may be based on waveforms stored in data associated with dongle 508 or NIEBS generator 509, such data may be updated or changed. FIG. 2 provides more details for the waveforms.

The user of the NIEBS treatment has a measure of control over the NIEBS treatment. For example, the user may be able to control the intensity or amplitude, the duration of the treatment, pulse duration, the pulse trains, periods or pauses between pulse trains, level of chaoticness, etc. Such control may be accomplished via buttons, sliders, wheels or other controls. For example, in an embodiment where 530 is an off the shelf device used in conjunction with dongle 508 then the controls for the NIEBS treatment may be physical controls on dongle 508. Dongle 508 may record levels or changes in level of control received at the dongle. This record may be output as the NIEBS Control Profile. Additionally, dongle 508 may have a display that displays the level of control of each variable to the user. The user may manually records preferred or desired levels of control to create a NIEBS Control Profile.

In one embodiment, the device 530 employs the built in NIEBS generator 509. In such an embodiment, the controls for the NIEBS generator may be physical controls on the device such as buttons or may be controlled via a graphical user interface associated with 530. Device 530 may be capable of generating a NIEBS Control Profile based on user control levels and uploading the NIEBS Control Profile to 540.

With reference now to FIG. 1B which is a graphical depiction of a NIEBS Control Profile 503. FIG. 1B depicts three variables that may be controlled 531, 532, and 533. For example, 531 may represent intensity, 532 may represent the length of pauses between pulse trains, and 533 may represent the level of chaoticness in generating the pulses. The shaded areas represent the value of the control. For example, intensity 531 is depicted as being at the value of 10. NIEBS Control Profile 503 may be associated with a touchscreen interface where a user can simply touch the shaded region and slide it up or down to adjust the value of the control. NIEBS Control Profile 503 may also be a simple depiction that may be shared with others. Depictions of NIEBS Control Profile may take on a number of forms and may simply be numerical representations. NIEBS Control Profile 503 is not limited to three controls but may have any number of controls.

### Braingear Ecosystem

With reference to FIG. 1C which is a depiction of an ecosystem for managing data associated with NIEBS. FIG. 1C relates to a Braingear Ecosystem. Braingear center 540 may be the same as depicted in FIG. 1A. The Braingear center 540 is connected to NIEBS program library which comprises a plurality of data files for use with NIEBS such as NIEBS data programs for the use by a NIEBS signal generator or may comprise NIEBS Control Profiles. Component 518 (Medical professional services) is connected to Braingear center 540 to manage prescriptions of NIEBS data programs that may be employed by users. Prescriptions may be prescribed by a healthcare professional and Braingear center 540 may be employed to manage the prescriptions. Component 519 is therapy manager for trials and feedback. For example, users of NIEBS may provide feed back to Braingear center 540 regarding the ratings or reviews of a given NIEBS treatment. Component 519 (Therapy manager trials and feedback) may also make suggestions for a NIEBS therapy. User database 520 tracks users associated with Braingear center 540. User forum and blog 521 allows user to interact with one another to review or suggest NIEBS treatments or exchange other data such as NIEBS Control Profiles. Components 519 (Therapy manager trials and feedback) and 521 (User forum and blog) may be employed to present users with recommendation for NIEBS treatments and NIEBS Control Profiles. For example, a NIEBS treatment or a NIEBS Control Profile favored by a celebrity may be endorsed and recommended via components 519 (Therapy manager trials and feedback) and 521 (User forum and blog). User 501 may connect with Braingear center 540 via Internet 511.

With reference to FIG. 2 which depicts wave forms that may be employed for use with the present technology. A NIEBS generator may receive wave forms from an audio source or from a waveform synthesizer associated with the NIEBS generator. The NIEBS generator may generate a NIEBS signal with associated wave forms for the NIEBS treatment. FIG. 2 depicts well known square wave forms for use in the present technology. The present technology is not limited to wave forms in FIG. 2 but may also employ other wave forms such as sine waves.

Wave forms for the present technology may be stored in a library and are used to create pulse patterns or pulse trains for use in NIEBS. The wave forms may be implemented via a programmable D/A converter. Research indicates that different pulse patterns have different effects on the brain, and that some pulse patterns have different effects on various conditions. Therefore, there is a need for a library of different pulse patterns to suit different health conditions.

The rate of pulses per second refers to a start of positive-going pulse to stop, with the delay until the next positive-going pulse starts. Like a sine wave, regardless of whether or not there is a negative-going pulse. “Beginning of a pulse rising, to the next time the pulse starts rising again.” The following are examples of pulse rates that may be employed by the present technology:

1. Pulse rate in range of 3-5 Hz. Low Freq.

2. Pulse rate in range of 50-100 Hz. Low Freq.

3. Pulse rate in range from 100-640 Hz. High Freq.

4. Pulse rate in range of 0.1-100 Hz

5. Direct current

Current level delivered: 1.5 mA. [milli-Ampere]

Current density on the skin: safety limit is between 25 and 60 microA/cm² [from Poreisz et al., 2007] The electric field across the brain tissue is on the order of less than 5 mV/mm, or 5 milli-Volts/millimeter.

Pulse pattern may be a Random Noise Stimulation pattern. Good results reported by Fertonani et al in paper “Random Noise Stimulation Improves Neuroplasticity in Perceptual Learning,” The Journal of Neuroscience, Oct. 26, 2011 31(43):15416-15423.

Noninvasive electrical brain stimulation (herein referred to as NIEBS) applies gentle micro-current pulses to the brain using electrodes. The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

In NIEBS significant amounts of current pass the skull and reach cortical and subcortical structures. In addition, depending on the montage, induced currents at subcortical areas, such as midbrain, pons, thalamus and hypothalamus are of similar magnitude than that of cortical areas. Incremental variations of electrode position on the head surface also influence which cortical regions are modulated. The high-resolution modeling predictions suggest that details of electrode montage influence current flow through superficial and deep structures. Also, laptop based methods for tPCS dose design using dominant frequency and spherical models. These modeling predictions and tools are the first step to advance rational and optimized use of tPCS and NIEBS.

It is widely accepted that NIEBS stimulates the brain to manufacture neurotransmitters, like endorphins, which improve moods, emotions and cognitive capabilities. Noninvasive electrical brain stimulation has also been proposed for treatment following a stroke, brain trauma, high blood pressure, and Alzheimer's disease, as well as any or all neurological disorders, any or all mental disorders, and any or all cognitive enhancements. The present technology may also be used by healthy users or users who are not suffering from any diagnosed disorders or diseases. For example, a healthy user may be a student using the present technology to increase focus and learning abilities or may be an athlete using the present technology to increase sports performance.

The signals apparently normalize the electrical output of the brain. NIEBS has thus been used or tested to treat substance dependence, depression and anxiety. It has been noted in at least some instances that NIEBS has equal or greater efficacy for the treatment of depression when compared to antidepressant medications, with fewer side effects. NIEBS may be used specifically in combination with anti-depressant drugs and may be used to eliminate the side effects of central nervous system (CNS) medications or drugs in general. NIEBS may also be used in conjunction with other traditional medicine.

Treatments can be used in association with the present technology in ranges from less than one second up to an infinite number of seconds. The present technology is not limited to a particular range of duration, current, or frequency. The following ranges are meant as examples and do not limit the present technology. In one embodiment, a range is used from 10 to 30 minutes in duration although the treatments may extend up to 11/2 hours depending on the electrical current configuration. The currents employed may be applied in pulse form or direct form with a pulse width in the range of from about 1 to about 500 milliseconds (ms) at a frequency of from about 0.1 Hertz (Hz) up to 1000 Hz with the current being less than 1 milliampere (mA) up to 5 mA

In accordance with an embodiment of the invention there is provided equipment for the implementation of a method as defined above, said equipment comprising a noninvasive electrical brain stimulation pulse generator and associated electrodes for applying pulses generated by the pulse generator to the head of a patient, wherein the equipment includes multiple electrodes.

In an embodiment of the invention, there is an audio signal player and at least one associated loudspeaker for converting output from the signal player into audible sound. The at least one loudspeaker is preferably a pair of earphones and the noninvasive electrical brain stimulation pulse generator and sound signal generator may be built into a single unit, but are not necessarily thus combined.

Note that there are the following types of stimulation configurations:

1. Positive going pulse, with a direct current average in one direction. Class 1A and Class 1B deliver a varying amount of direct current in little bursts.

2. Alternating current pulses, where the direction of current alternates from positive going to negative going, as in Class IIA and Class IIB and IIC and IID. The average may be in one direction predominantly, or may average out to zero if the pulses are symmetric and equal in duration over time. You can see that for some modes, there is a net direct current passing thru the brain.

3. Class III shows a pulse train with a delay between delivery of a series of pulses.

The next paragraphs discuss how this delay may be configured, and is part of the overall therapy formulation that is available to a medical practitioner.

1. Random time period. Use a random number generator with a specified range in seconds. For example, 1-100 seconds. Run the random number generator which is set to produce a number between 1 and 100. Use that number as the time period between pulses. Run the generator after each pulse to determine the next time delay, or period, from the last pulse.

2. Semi-random time period.

Pick some time periods that are known to have some therapeutic effect. Make a table. For example:

Random No. 1 3 5 10 20 40 60 100.

Bin containing 1 2 3 4 5 6 7 8

the delay

Then randomly select from this group of time periods. Again, use a random number generator whose bounds are the number of allowed states. In the above example, there are 8 possible delay time periods. Set the random number generator to select any of the numbers from 1 to 8. Use the time delay associated with that bin number.

Say the random number generator picks 4. That means we use 10 second delay as the time period to the next pulse train initiation.

3. Periodic but increasing delay, with a plan

Here the time delay from one pulse train event to the next is arbitrarily set to predetermined sequence. It may be one with a set increase from one period duration to the next. As in 5 10 30 60 repeat 5 10 30 60.

4. Periodic, static period

Set delay to one of the group [1, 2, 3, 4, 5, 6, 7, 8, 9, 10] seconds. Or any other time period from 1 to 300 seconds, for example.

5. Continuous pulse train with no delay between any arbitrary group of pulses. Arbitrary duration of such pulse trains, selected from group [1-1000] seconds.

6. Direct Current Stimulation

No pulses, just application of a constant voltage for some time period. One could consider this a special case of a single positive going pulse with a really long time duration.

Notes on using chaotic/random pulse for NIEBS:

Pulses or pulse trains for NIEBS may be patterned or random. However, the idea of random pulses may not be desirable as random may still indicate a measureable structure impulse. The term chaotic pattern is better description of the pulse referred to herein. Chaotic may also be used to define the variety of the pauses or periods in between pulse trains. The level of chaoticness may be controlled via a controller similar to 109 of FIG. 1A.

### Computer Implemented Methods

It should be appreciated that the methods described herein may be computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of computer usable storage medium. In one embodiment, the methods may reside in a computer usable storage medium having instructions embodied therein that when executed cause a computer system to perform the method. In one embodiment, the NIEBS signals described herein are non-transitory but rather are sent over wired connections to the electrodes.

It is intended that the foregoing detailed description be regarded as illustrative rather than limiting, and that it be understood that the detailed description should not be used to limit the scope of the invention.

1. A computer-implemented method for sharing information related to a noninvasive electrical brain stimulation “NIEBS” treatment, comprising:
providing a NIEBS treatment to a user via a NIEBS generator, said NIEBS treatment having variables;
receiving adjustments to the variables of said NIEBS treatment via user input; and
creating a NIEBS Control Profile based on said received adjustments.

2. The method of claim 1 further comprising sharing said NIEBS Control Profile.

3. The method of claim 2, wherein sharing said NIEBS Control Profile comprises uploading said NIEBS Control Profile to a server.

4. The method of claim 1, wherein the NIEBS Control Profile includes adjustments to at least one of:
intensity or amplitude;
the duration of the treatment,
pulse duration,
the pulse trains,
periods or pauses between pulse trains, and
level of chaotic behaviour.

5. The method of claim 2 wherein said sharing said NIEBS Control Profile comprises sharing a review of said NIEBS Control Profile.

6. The method of claim 1 wherein said NIEBS generator is a dongle associated with an off the shelf device.

7. The method of claim 1 wherein said NIEBS generator is incorporated into a smartphone.

8. A computer-implemented method for sharing information related to a noninvasive electrical brain stimulation “NIEBS” treatment, comprising:
receiving a NIEBS Control Profile at a server;
making said NIEBS Control Profile available to users for download;
receiving information from a user relating to said NIEBS Control Profile; and
making said information from said user available with said NIEBS Control Profile.

9. The method according to claim 8, further comprising providing a recommendation for a NIEBS Control Profile.

10. The method of claim 1, wherein the NIEBS Control Profile includes adjustments to at least one of:
intensity or amplitude;
the duration of the treatment,
pulse duration,
the pulse trains,
periods or pauses between pulse trains, and
level of chaotic behaviour.

11. A computer-readable medium comprising computer-readable instructions to perform the method of claim 1.

12. An apparatus configured to perform the method of claim 1.

13. A method for sharing information related to a NIEBS treatment, comprising:
providing a NIEBS treatment to a user via a NIEBS generator;
receiving adjustments to variables of said NIEBS via user controls; and
creating a NIEBS Control Profile based on said adjustments.

14. The method of claim 13 further comprising sharing said NIEBS Control Profile.

15. The method of claim 14 wherein said sharing said NIEBS Control Profile comprises sharing a review of said NIEBS Control Profile.

16. The method of claim 13 wherein said NIEBS generator is a dongle associated with an off the shelf device.

17. The method of claims 13 wherein said NIEBS generator is incorporated into a smartphone.

18. A method for sharing information related to a NIEBS treatment, comprising:
receiving a NIEBS Control Profile at a server;
making said NIEBS Control Profile available to users for download;
receiving feedback from said users regarding said NIEBS Control Profile; and
making said feedback from said user available with said NIEBS Control Profile.
